(19) United States
(12) Patent Application Publication
(10) Pub. No.: US 2014/0326242 A1
(43) Pub. Date: Nov. 6, 2014

(54) SYSEMS AND METHODS FOR USING PARTIAL CO2 REBREATHING INTEGRATED IN A VENTILATOR AND MEASUREMENTS THEREOF TO DETERMINE NONINVASIVE CARDIAC OUTPUT

(71) Applicant: KONINKLIJKE PHILIPS N.V., EINDHOVEN (NL)

(72) Inventors: Samir Ahmad, San Diego, CA (US); Smita Garde, Carlsbad, CA (US)

(73) Assignee: Koninklijke Philips N.V., Eindhoven (NL)

(21) Appl. No.: 14/360,995

(22) PCT Filed: Nov. 15, 2012
(86) PCT No.: PCT/IB2012/056447

§ 371 Date: May 28, 2014

Related U.S. Application Data
(60) Provisional application No. 61/565,539, filed on Dec. 1, 2011.

(51) Int. Cl. A61M 16/00; A61M 16/08; A61B 5/00; A61M 16/20; A61B 5/0205

(52) U.S. Cl. CPC A61M 16/0045; A61M 16/0051; A61M 16/205; A61M 16/0069; A61M 16/0003; A61B 5/0205; A61B 5/7278; A61B 5/4836; A61M 16/0883; A61B 5/0836
USPC 128/204.23; 128/204.21

(57) ABSTRACT

Systems and methods for providing a ventilator for partial CO₂ rebreathing using exhaust valves integrated in a ventilator system to increase a CO₂ rebreathing volume of a subject. Non-invasive measurements of CO₂ parameters and partial CO₂ rebreathing are used to determine noninvasive cardiac output parameters of the subject.

### 1. FIELD

The present disclosure pertains to systems and methods for providing non-invasive cardiac output measurements of a subject using partial CO₂ rebreathing, integrated in a ventilation system that is configured to use integrated valves, including an exhalation valve, flow control valve and safety valve, to increase the rebreathing volume of a subject.

### 2. DESCRIPTION OF THE RELATED ART

It is well known that some types of respiratory therapy involve mechanical ventilation. It is well known that some types of respiratory therapy involve the delivery of a flow of breathable gas to the airway of a subject. It is known that (partial) rebreathing may affect one or more CO₂ parameters of the flow of breathable gas delivered to a subject. It is well known that the proper administration of respiratory therapy hinges on having accurate and up-to-date information regarding a variety of medical information pertaining to the subject, including but not limited to the lung mechanics of a subject and/or the cardiac output of a subject.

### SUMMARY

Accordingly, it is an object of one or more embodiments of the present invention to provide a ventilator system. The ventilator system comprises a pressure generator configured to generate a pressurized flow of breathable gas for delivery to an airway of a subject; a delivery circuit configured to guide the pressurized flow of breathable gas from the pressure generator to the airway of the subject; a first exhaust valve in fluid communication with the delivery circuit at a first exhaust point, the first exhaust valve being configured to selectively exhaust gas from the delivery circuit at the first exhaust point; a second exhaust valve in fluid communication with the delivery circuit at a second exhaust point, the second exhaust valve being configured to selectively exhaust gas from the delivery circuit at the second exhaust point, and wherein the delivery circuit between the first exhaust point and the second exhaust point has a rebreathing storage capacity; and one or more processors configured to execute processing modules. The processing modules comprise a control module configured to control operation of the ventilator system in a first therapy mode or a second therapy mode; and a valve control module configured to control the first exhaust valve and the second exhaust valve to exhaust exhaled gas from the delivery circuit, wherein the valve control module is configured such that (i) during operation of the ventilator system in a first therapy mode, exhaled gas is exhausted from the delivery circuit primarily through the second exhaust valve, and (ii) during operation of the ventilator system in a second therapy mode, exhaled gas is exhausted from the delivery circuit primarily through the first exhaust valve, thereby increasing a rebreathing volume of the subject and the delivery circuit by the rebreathing storage capacity responsive to the ventilator system being operated in the second therapy mode. The first therapy mode may be referred to herein as a default ventilation therapy mode. The second therapy mode may be referred to herein as a rebreathing therapy mode.

It is yet another aspect of one or more embodiments of the present invention to provide a method for providing ventilation to an airway of a subject through a ventilation system. The method comprises generating a pressurized flow of breathable gas for delivery to the airway of the subject; guiding the pressurized flow of breathable gas to the airway of the subject via a delivery circuit; determining whether the ventilation system is operating in a first therapy mode or a second therapy mode; responsive to a determination that the ventilation system is operating in the second therapy mode, selectively exhausting gas from the delivery circuit primarily via a first exhaust point; and responsive to a determination that the ventilation system is operating in the first therapy mode, selectively exhausting gas from the delivery circuit primarily via a second exhaust point, wherein the delivery circuit has a rebreathing storage capacity between the first exhaust point and the second exhaust point, wherein selectively exhausting gas from the delivery circuit primarily via the first exhaust point, responsive to the determination that the ventilation system is operating in the second therapy mode, increases a rebreathing volume of the subject and the delivery circuit by the rebreathing storage capacity.

It is yet another aspect of one or more embodiments to provide a system configured to provide ventilation to an airway of a subject through a ventilation system. The system comprises means for generating a pressurized flow of breathable gas for delivery to the airway of the subject; delivery means guiding the pressurized flow of breathable gas to the airway of the subject; means for determining whether the ventilation system is operating in a first therapy mode or a second therapy mode; first exhaust means for selectively exhausting gas from the delivery circuit primarily via a first exhaust point, responsive to a determination that the ventilation system is operating in the second therapy mode; and means for selectively exhausting gas from the delivery circuit primarily via a second exhaust point, responsive to a determination that the ventilation system is operating in the first therapy mode, wherein the delivery means has a rebreathing storage capacity, wherein operation of the first exhaust means increases a rebreathing volume of the subject and the delivery circuit by the rebreathing storage capacity.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 schematically illustrates a system configured to provide ventilation to the airway of a subject through a ventilation system, according to certain embodiments; and

FIG. 2 illustrates a method for providing ventilation to the airway of a subject through a ventilation system, according to certain embodiments.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled to move as one while maintaining a constant orientation relative to each other.

As used herein, the word “unitary” means a component is created as a single piece or unit. That is, a component that includes pieces that are created separately and then coupled as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

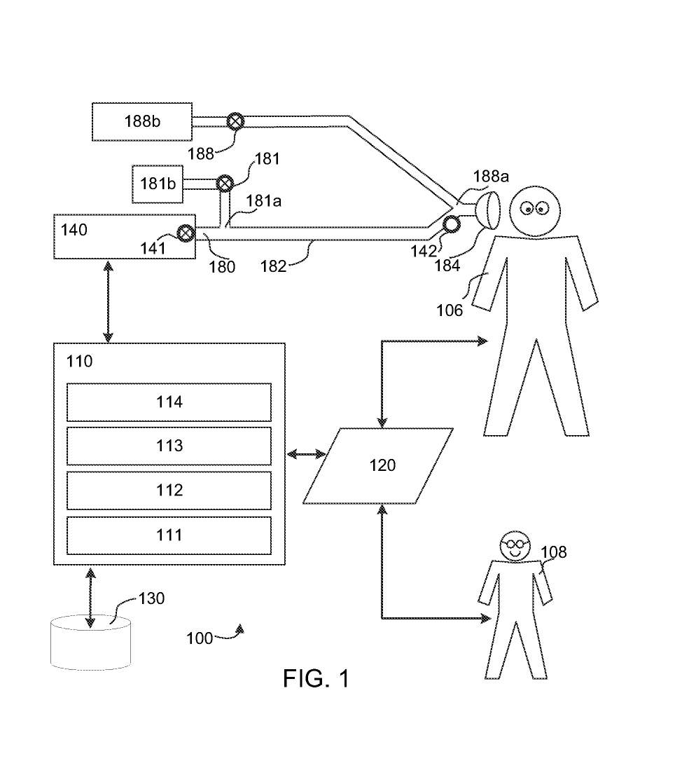

FIG. 1 schematically illustrates a ventilation system 100 configured to provide ventilation to the airway of a subject 106. Ventilation system 100 may simply be referred to as system 100. System 100 may be implemented as, integrated with, and/or operating in conjunction with a respiratory therapy device. System 100 uses partial CO₂ rebreathing, as well as measurements of CO₂ parameters related thereto, to determine cardiac output of a subject in a non-invasive manner. Additional information on using (partial) CO₂ rebreathing to determine cardiac output non-invasively may be found in U.S. patent application Ser. No. 10/424,656, entitled “Methods For Inducing Temporary Changes In Ventilation For Estimation Of Hemodynamic Performance,” and filed Apr. 28, 2003, which is hereby incorporated by reference into the present application in its entirety.

System 100 may include one or more of a pressure generator 140, a delivery circuit 180, a first exhaust valve 181, a second exhaust valve 188, one or more sensors 142, an electronic storage 130, a user interface 120, a processor 110, a control module 111, a valve control module 112, a parameter determination module 113, a timing module 114, and/or other components.

Pressure generator 140 of system 100 in FIG. 1 may be integrated, combined, or connected with a ventilator and/or (positive) airway pressure device (PAP/CPAP/BiPAP®/etc.) and configured to provide a pressurized flow of breathable gas for delivery to the airway of subject 106, e.g. via delivery circuit 180. Delivery circuit 180 may sometimes be referred to as subject interface 180. Subject 106 may or may not initiate one or more phases of respiration. Ventilation therapy may be implemented as pressure control, pressure support, and/or volume control. For example, to support inspiration, the pressure of the pressurized flow of breathable gas may be adjusted to an inspiratory pressure. Alternatively, and/or simultaneously, to support expiration, the pressure and/or flow of the pressurized flow of breathable gas may be adjusted to an expiratory pressure. Other schemes for providing respiratory support through the delivery of the pressurized flow of breathable gas are contemplated. Pressure generator 140 may be configured to adjust pressure levels, flow, humidity, velocity, acceleration, and/or other parameters of the pressurized flow of breathable gas in substantial synchronization with the breathing cycle of the subject.

A pressurized flow of breathable gas may be delivered from pressure generator 140 to the airway of subject 106 via a delivery circuit 180. Delivery circuit 180 may include a conduit 182 and/or a subject interface appliance 184. Conduit 182 may include a flexible length of hose, or other conduit, either in single-limb or dual-limb configuration that places subject interface appliance 184 in fluid communication with pressure generator 140. Conduit 182 forms a flow path through which the pressurized flow of breathable gas is communicated between subject interface appliance 184 and pressure generator 140. In some embodiments, pressure generator may include an inhalation valve 141. Inhalation valve 141 may be a controllable and/or adjustable valve. Inhalation valve 141 may be incorporated into pressure generator 140, delivery circuit 180, and/or another component of system 100.

Subject interface appliance 184 of system 100 in FIG. 1 is configured to deliver the pressurized flow of breathable gas to the airway of subject 106. As such, subject interface appliance 184 may include any appliance suitable for this function. In one embodiment, pressure generator 140 is a dedicated ventilation device and subject interface appliance 184 is configured to be removably coupled with another interface appliance being used to deliver respiratory therapy to subject 106. For example, subject interface appliance 184 may be configured to engage with and/or be inserted into an endotracheal tube, a tracheotomy portal, and/or other interface appliances. In one embodiment, subject interface appliance 184 is configured to engage the airway of subject 106 without an intervening appliance. In this embodiment, subject interface appliance 184 may include one or more of an endotracheal tube, a nasal cannula, a tracheotomy tube, a nasal mask, a nasal/oral mask, a full-face mask, a total facemask, and/or other interface appliances that communicate a flow of gas with an airway of a subject. The present disclosure is not limited to these examples, and contemplates delivery of the pressurized flow of breathable gas to subject 106 using any subject interface.

First exhaust valve 181 of system 100 in FIG. 1 is configured to be in fluid communication with delivery circuit 180 at a first exhaust point 181a. First exhaust valve 181 may be configured to exhaust gas selectively from delivery circuit 180 at first exhaust point 181a. During typically usage, first exhaust valve 181 may be used to exhaust gas from delivery circuit 180 responsive to pressure rising unexpectedly (e.g., above a threshold level) to provide relief for subject 106 and/or pressure generator 140 from excess pressure. First exhaust valve 181 may be in fluid communication with first exhaust circuit 181b, which may include, e.g., an exhaust filter, and/or other components. First exhaust valve 181 may fluidly couple delivery circuit 180 to ambient air, or to an inlet of pressure generator 140, and/or to another component of system 100.

Second exhaust valve 188 of system 100 in FIG. 1 is configured to be in fluid communication with delivery circuit 180 at a second exhaust point 188a. Second exhaust valve 188 may be configured to exhaust gas selectively from delivery circuit 180 at second exhaust point 188a. In some embodiments and/or configurations, second exhaust point 188a may be located in the expiratory limb of the delivery circuit 180. In some other embodiments and/or configurations, second exhaust point 188a may be located along delivery circuit 180 between the airway of subject 106 and first exhaust point 181a. A section of delivery circuit 180 between first exhaust point 181a and second exhaust point 188a has a rebreathing storage capacity. The rebreathing storage capacity may be referred to as a rebreathing storage volume. The rebreathing storage capacity may be similar to the storage capacity of four feet to six feet of single-limb or dual-limb hose, and/or another amount of standard-sized 22 mm, 15 mm or 10 mm diameter hose. In some implementations, the rebreathing storage capacity may be at least about 200 ml to about 1500 ml, and/or another capacity. Second exhaust valve 188 may be referred to as an exhalation valve. Second exhaust valve 188 may be in fluid communication with second exhaust circuit 188b, which may include, e.g., an exhaust filter, and/or other components. Second exhaust valve 188 may fluidly couple delivery circuit 180 to ambient air, or to an inlet of pressure generator 140 via conduit 182, or to subject interface appliance 184, and/or to another component of system 100.

In some embodiments, first exhaust valve 181 is closed when system 100 is operating in the default ventilation therapy mode. In some embodiments, when system 100 is operating in the default ventilation therapy mode, exhaled gas is exhausted from delivery circuit 180 primarily through second exhaust valve 188.

In some embodiments, first exhaust valve 181 is controlled such that, during operation of system 100 in the rebreathing therapy mode, exhaled gas is exhausted from delivery circuit 180 primarily through first exhaust valve 181, thereby increasing the rebreathing volume of subject 106 and delivery circuit 180 by the rebreathing storage capacity of delivery circuit 180 between first exhaust point 181a and second exhaust point 188a. During inhalation phases in the rebreathing therapy mode, subject 106 inhales previously exhaled gas that was stored in the rebreathing volume, e.g. in the rebreathing storage capacity. Note that rebreathing may thus be accomplished without the addition of a separate and discrete rebreathing loop to system 100. In some embodiments, during operation of system 100 in the rebreathing therapy mode, first exhaust valve 181 is opened in the exhalation phase.

Electronic storage 130 of system 100 in FIG. 1 comprises electronic storage media that electronically stores information. The electronic storage media of electronic storage 130 may include one or both of system storage that is provided integrally (i.e., substantially non-removable) with system 100 and/or removable storage that is removably connectable to system 100 via, for example, a port (e.g., a USB port, a FireWire port, etc.) or a drive (e.g., a disk drive, etc.). Electronic storage 130 may include one or more of optically readable storage media (e.g., optical disks, etc.), magnetically readable storage media (e.g., magnetic tape, magnetic hard drive, floppy drive, etc.), electrical charge-based storage media (e.g., EPROM, EEPROM, RAM, etc.), solid-state storage media (e.g., flash drive, etc.), and/or other electronically readable storage media. Electronic storage 130 may store software algorithms, information determined by processor 110, information received via user interface 120, and/or other information that enables system 100 to function properly. For example, electronic storage 130 may record or store one or more gas and/or respiratory parameters (as discussed elsewhere herein), one or more CO₂ parameters, one or more cardiac output parameters, and/or other information. Electronic storage 130 may be a separate component within system 100, or electronic storage 130 may be provided integrally with one or more other components of system 100 (e.g., processor 110).

User interface 120 of system 100 in FIG. 1 is configured to provide an interface between system 100 and a user (e.g., user 108, subject 106, a caregiver, a therapy decision-maker, etc.) through which the user can provide information to and receive information from system 100. This enables data, results, and/or instructions and any other communicable items, collectively referred to as “information,” to be communicated between the user and system 100. An example of information that may be conveyed to user 108 is a report detailing the changes in one or more determined CO₂ parameters of subject 106 throughout a period during which the subject is receiving (multiple modes of) therapy. Examples of interface devices suitable for inclusion in user interface 120 include a keypad, buttons, switches, a keyboard, knobs, levers, a display screen, a touch screen, speakers, a microphone, an indicator light, an audible alarm, and a printer. Information may be provided to user 108 or subject 106 by user interface 120 in the form of auditory signals, visual signals, tactile signals, and/or other sensory signals.

It is to be understood that other communication techniques, either hard-wired or wireless, are also contemplated herein as user interface 120. For example, in one embodiment, user interface 120 may be integrated with a removable storage interface provided by electronic storage 130. In this example, information is loaded into system 100 from removable storage (e.g., a smart card, a flash drive, a removable disk, etc.) that enables the user(s) to customize the implementation of system 100. Other exemplary input devices and techniques adapted for use with system 100 as user interface 120 include, but are not limited to, an RS-232 port, RF link, an IR link, modem (telephone, cable, Ethernet, internet or other). In short, any technique for communicating information with system 100 is contemplated as user interface 120.

One or more sensors 142 of system 100 in FIG. 1 are configured to generate output signals conveying measurements related to parameters of respiratory airflow and/or airway mechanics. These parameters may include one or more of flow, (airway) pressure, humidity, velocity, acceleration, and/or other parameters. Sensor 142 may be in fluid communication with conduit 182 and/or subject interface appliance 184. Sensor 142 may generate output signals related to physiological parameters pertaining to subject 106. In some embodiments, one or more sensors 142 may include one or more CO₂ sensors.

The illustration of sensor 142 including a single member in FIG. 1 is not intended to be limiting. The illustration of sensor 142 at or near subject interface appliance 184 is not intended to be limiting. In one embodiment sensor 142 includes a plurality of sensors operating as described above by generating output signals conveying information related to parameters associated with the state and/or condition of an airway of subject 106, the breathing of subject 106, the gas breathed by subject 106, the composition of the gas breathed by subject 106, one or more CO₂ parameters of the gas breathed by subject 106, the delivery of the gas to the airway of subject 106, and/or a respiratory effort by the subject. The one or more CO₂ parameters and/or measurements may include, without limitation, end-tidal CO₂ measurements, volumetric CO₂ measurements, mixed-venous CO₂ measurements, arterial CO₂ measurements, and/or other CO₂ parameters and/or measurements. For example, a parameter may be related to a mechanical unit of measurement of a component of pressure generator 140 (or of a device that pressure generator 140 is integrated, combined, or connected with) such as valve drive current, rotor speed, motor speed, blower speed, fan speed, or a related measurement that may serve as a proxy for any of the previously listed parameters through a previously known and/or calibrated mathematical relationship. Resulting signals or information from sensor 142 may be transmitted to processor 110, user interface 120, electronic storage 130, and/or other components of system 100. This transmission may be wired and/or wireless.

Processor 110 of system 100 in FIG. 1 is configured to provide information processing capabilities in system 100. As such, processor 110 includes one or more of a digital processor, an analog processor, a digital circuit designed to process information, an analog circuit designed to process information, a state machine, and/or other mechanisms for electronically processing information. Although processor 110 is shown in FIG. 1 as a single entity, this is for illustrative purposes only. In some implementations, processor 110 includes a plurality of processing units.

As is shown in FIG. 1, processor 110 is configured to execute one or more computer program modules. The one or more computer program modules include one or more of control module 111, valve control module 112, parameter determination module 113, timing module 114, and/or other modules. Processor 110 may be configured to execute modules 111, 112, 113, and/or 114 by software; hardware; firmware; some combination of software, hardware, and/or firmware; and/or other mechanisms for configuring processing capabilities on processor 110.

It should be appreciated that although modules 111, 112, 113, and 114 are illustrated in FIG. 1 as being co-located within a single processing unit, in implementations in which processor 110 includes multiple processing units, one or more of modules 111, 112, 113, and/or 114 may be located remotely from the other modules. The description of the functionality provided by the different modules 111, 112, 113, and/or 114 described below is for illustrative purposes, and is not intended to be limiting, as any of modules 111, 112, 113, and/or 114 may provide more or less functionality than is described. For example, one or more of modules 111, 112, 113, and/or 114 may be eliminated, and some or all of its functionality may be provided by other ones of modules 111, 112, 113, and/or 114. Note that processor 110 may be configured to execute one or more additional modules that may perform some or all of the functionality attributed below to one of modules 111, 112, 113, and/or 114.

Parameter determination module 113 of system 100 in FIG. 1 is configured to determine one or more gas parameters, breathing parameters, and/or other parameters from output signals generated by sensor(s) 142. The one or more gas parameter may include and/or be related to one or more of (peak) flow, flow rate, (tidal) volume, pressure, temperature, humidity, velocity, acceleration, gas composition (e.g. concentration(s) of one or more constituents such as, e.g., CO₂), thermal energy dissipated, (intentional) gas leak, and/or other measurements related to the (pressurized) flow of breathable gas. One or more breathing parameters may be derived from gas parameters and/or other output signals conveying measurements of the pressurized flow of breathable gas. The one or more breathing parameters may include one or more of respiratory rate, breathing period, inhalation time or period, exhalation time or period, respiration flow curve shape, transition time from inhalation to exhalation and/or vice versa, transition time from peak inhalation flow rate to peak exhalation flow rate and/or vice versa, respiration pressure curve shape, maximum proximal pressure drop (per breathing cycle and/or phase), and/or other breathing parameters. Some or all of this functionality may be incorporated, shared, and/or integrated into other computer program modules of processor 110.

In some embodiments, parameter determination module 113 may be configured to determine one or more cardiac output parameters of subject 106. The one or more cardiac output parameters may be based on measurements of CO₂ present in delivery circuit 180 (e.g., of concentration, relative level, and/or other measurements). For example, while subject 106 is undergoing respiratory therapy in a first therapy mode, such as the default ventilation therapy mode described above, a first measurement (or set of measurements) of CO₂ present in delivery circuit 180 is determined by parameter determination module 113. Subsequently, subject 106 may undergo respiratory therapy in a second therapy mode, such as the rebreathing therapy mode described above, or another (partial) rebreathing therapy mode. Parameter determination module 113 may determine a measurement (or set of measurements) of CO₂ present in delivery circuit 180 during operation in the second therapy mode. Due to the change in the rebreathing volume between the first therapy mode and the second therapy mode, it is expected that the amount, concentration, and/or level of CO₂ will be elevated in the second therapy mode. The difference between the measurements taken during the first therapy mode and the second therapy mode can then be used to determine one or more cardiac output parameters. Measurements taken during the first and second therapy modes may be referred to as differential CO₂ measurements. In some embodiments, subject 106 may undergo respiratory therapy in more than two different therapy modes. In some embodiments, transitions between different therapy modes may be caused after individual breaths, after a plurality of breaths, after a predetermined time period of, e.g., 30 seconds, one minute, two minutes, three minutes, and/or other time periods, and/or after one or more other predetermined events or occurrences.

Timing module 114 is configured to determine whether a current respiratory phase is an inhalation phase or an exhalation phase. In some embodiments, timing module 114 may be configured to determine respiratory timing parameters and/or other timing parameters related to the operation of system 100, such as transitions in breathing between inhalations and exhalations. Respiratory timing parameters may include transitional moments that separate inhalation phases from exhalation phases and/or vice versa, breathing period, respiratory rate, inhalation time or period, exhalation time or period, start and/or end of inhalation phases, start and/or end of exhalation phases, and/or other respiratory timing parameters. One or more determinations by timing module 114 may be used, shared, and/or incorporated in other components of system 100.

Control module 111 is configured to control operation of system 100 in the first therapy mode, the second therapy mode, and/or other therapy modes. Control module 111 may be configured to control transitions between different therapy modes. Control module 111 may be configured to determine what the current therapy mode is, and/or share such information with other components of system 100. Control module 111 may be configured to control pressure generator 140 such that one or more gas parameters of the pressurized flow of breathable gas are varied over time in accordance with a respiratory therapy regimen. Control module 111 may be configured to control pressure generator 140 to provide the pressurized flow of breathable gas at inhalation pressure levels during inhalation phases, and at exhalation pressure levels during exhalation phases. Parameters determined by parameter determination module 113, timing module 114, and/or received through sensors 142 may be used by control module 111, e.g. in a feedback manner, to adjust one or more therapy modes/settings/operations of system 100. Alternatively, and/or simultaneously, signals and/or information received through user interface 120 may be used by control module 111, e.g. in a feedback manner, to adjust one or more therapy modes/settings/operations of system 100. Control module 111 may be configured to time its operations relative to the transitional moments in the breathing cycle of a subject, over multiple breath cycles, and/or in any other relation to any detected occurrences or determinations by timing module 114.

Valve control module 112 is configured to control first exhaust valve 181 and/or second exhaust valve 188. For example, during operation of system 100 in the default ventilation therapy mode, exhaled gas may be exhausted from delivery circuit 180 primarily through second exhaust valve 188. This may be accomplished, e.g., by opening second exhaust valve 188, while first exhaust valve 181 remains closed. During operation of system 100 in the rebreathing therapy mode, exhaled gas may be exhausted from delivery circuit 180 primarily through first exhaust valve 181. This may be accomplished, e.g., by opening first exhaust valve 181, while simultaneously fully or partially closing second exhaust valve 188. It is noted that controlling the first and second exhaust valve thus in the rebreathing therapy mode increases the rebreathing volume of subject 106 and/or delivery circuit 180 by the rebreathing storage capacity. During inhalation phases in the rebreathing therapy mode, subject 106 inhales previously exhaled gas, which may have a higher concentration of CO₂ than ambient air, that was stored in the rebreathing volume, e.g. in the rebreathing storage capacity. This may affect one or more CO₂ parameters of system 100, the composition of the inhaled gas by subject 106, physiological parameters of subject 106 pertaining to CO₂, and/or other parameters.

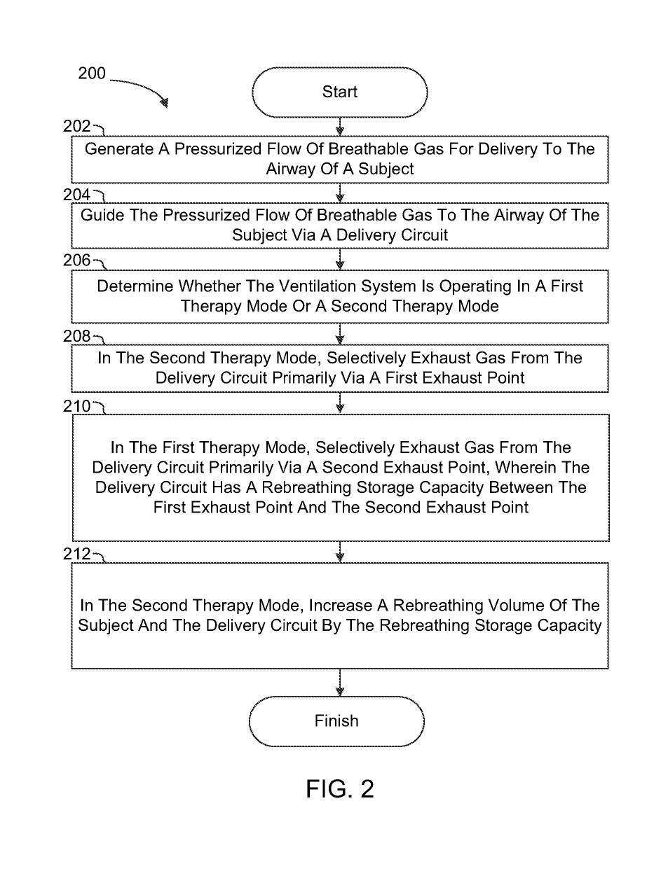

FIG. 2 illustrates a method for providing ventilation to the airway of a subject through a ventilation system. The operations of method 200 presented below are intended to be illustrative. In certain embodiments, method 200 may be accomplished with one or more additional operations not described, and/or without one or more of the operations discussed. Additionally, the order in which the operations of method 200 are illustrated in FIG. 2 and described below is not intended to be limiting.

In certain embodiments, method 200 may be implemented in one or more processing devices (e.g., a digital processor, an analog processor, a digital circuit designed to process information, an analog circuit designed to process information, a state machine, and/or other mechanisms for electronically processing information). The one or more processing devices may include one or more devices executing some or all of the operations of method 200 in response to instructions stored electronically on an electronic storage medium. The one or more processing devices may include one or more devices configured through hardware, firmware, and/or software to be specifically designed for execution of one or more of the operations of method 200.

At an operation 202, a pressurized flow of breathable gas is generated for delivery to the airway of a subject. In one embodiment, operation 202 is performed by a pressure generator similar to or substantially the same as pressure generator 140 (shown in FIG. 1 and described above).

At an operation 204, the pressurized flow of breathable gas is guided to the airway of a subject. In one embodiment, operation 204 is performed by a delivery circuit similar to or substantially the same as delivery circuit 180 (shown in FIG. 1 and described above).

At an operation 206, the current operating mode of the ventilation system is determined: first therapy mode or second therapy mode. In one embodiment, operation 206 is performed by a control module similar to or substantially the same as control module 111 (shown in FIG. 1 and described above).

At an operation 208, responsive to a determination that the ventilation system is operating in the second therapy mode, exhaled gas is exhausted from the delivery circuit primarily via a first exhaust point. In one embodiment, operation 208 is performed by an exhaust valve similar to or substantially the same as first exhaust valve 181 (shown in FIG. 1 and described above).

At an operation 210, responsive to a determination that the ventilation system is operating in the first therapy mode, exhaled gas is exhausted from the delivery circuit primarily via a second exhaust point, corresponding to a second exhaust valve. The first exhaust point and second exhaust point may be disposed on a conduit similar to or substantially the same as conduit 182 (shown in FIG. 1 and described above), such that the delivery circuit has a rebreathing storage capacity between the first exhaust point and the second exhaust point. In one embodiment, operation 210 is performed by an exhaust circuit similar to or substantially the same as second exhaust circuit 188 (shown in FIG. 1 and described above).

At an operation 212, responsive to a determination that the ventilation system is operating in the second therapy mode, a rebreathing volume of the subject and the delivery circuit is increased by the rebreathing storage capacity. In one embodiment, operation 212 is performed by exhaust valves similar to or substantially the same as first exhaust valve 181 and second exhaust valve 188 (shown in FIG. 1 and described above).

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A ventilator system comprising:
a pressure generator configured to generate a pressurized flow of breathable gas for delivery to an airway of a subject;
a delivery circuit configured to guide the pressurized flow of breathable gas from the pressure generator to the airway of the subject;
a first exhaust valve in fluid communication with the delivery circuit at a first exhaust point, the first exhaust valve being configured to selectively exhaust gas from the delivery circuit at the first exhaust point;
a second exhaust valve in fluid communication with the delivery circuit at a second exhaust point, the second exhaust valve being configured to selectively exhaust gas from the delivery circuit at the second exhaust point, and wherein the delivery circuit between the first exhaust point and the second exhaust point has a rebreathing storage capacity; and
one or more processors configured to execute processing modules, the processing modules comprising:
a control module configured to control operation of the ventilator system in a first therapy mode or a second therapy mode; and
a valve control module configured to control the first exhaust valve and the second exhaust valve to exhaust exhaled gas from the delivery circuit, wherein the valve control module is configured such that (i) during operation of the ventilator system in a first therapy mode, exhaled gas is exhausted from the delivery circuit primarily through the second exhaust valve, and (ii) during operation of the ventilator system in a second therapy mode, exhaled gas is exhausted from the delivery circuit primarily through the first exhaust valve, thereby increasing a CO₂ rebreathing volume of the subject and the delivery circuit by the rebreathing storage capacity responsive to the ventilator system being operated in the second therapy mode.

2. The ventilator system of claim 1, further comprising:
a timing module configured to determine whether a current respiratory phase is an inhalation phase or an exhalation phase,
wherein, responsive to the ventilator system being operated in the second therapy mode, during inhalation phases the first exhaust valve remains closed.

3. The ventilator system of claim 1, wherein, responsive to the ventilator system being operated in the second therapy mode, during exhalation phases the first exhaust valve is opened and the second exhaust valve is fully or partially closed.

4. The ventilator system of claim 1, further comprising:
one or more sensors including one or more CO₂ sensors configured to generate one or more output signals conveying information related to one or more gas parameters of the pressurized flow of breathable gas;
a parameter determination module configured to determine one or more CO₂ parameters based on output signals generated by the one or more CO₂ sensors during the first therapy mode and the second therapy mode.

5. The ventilator system of claim 4, wherein the parameter determination module is further configured to determine one or more cardiac output parameters based on the determined CO₂ parameters for partial CO₂ rebreathing.

6. A method for providing ventilation to an airway of a subject through a ventilation system, the method comprising;
generating a pressurized flow of breathable gas for delivery to the airway of the subject;
guiding the pressurized flow of breathable gas to the airway of the subject via a delivery circuit;
determining whether the ventilation system is operating in a first therapy mode or a second therapy mode;
responsive to a determination that the ventilation system is operating in the second therapy mode, selectively exhausting gas from the delivery circuit primarily via a first exhaust point;
responsive to a determination that the ventilation system is operating in the first therapy mode, selectively exhausting gas from the delivery circuit primarily via a second exhaust point, wherein the delivery circuit has a rebreathing storage capacity between the first exhaust point and the second exhaust point,
wherein selectively exhausting gas from the delivery circuit primarily via the first exhaust point, responsive to the determination that the ventilation system is operating in the second therapy mode, increases a CO₂ rebreathing volume of the subject and the delivery circuit by the rebreathing storage capacity.

7. The method of claim 6, further comprising:
determining whether a current respiratory phase of the subject is an inhalation phase or an exhalation phase,
wherein selectively exhausting gas from the delivery circuit via the first exhaust point and/or via the second exhaust point is furthermore responsive to a determination that the current respiratory phase of the subject is not an inhalation phase.

8. The method of claim 6, wherein selectively exhausting gas from the delivery circuit via the first exhaust point is performed during inhalation phases and exhalation phases.

9. The method of claim 6, further comprising:
generating one or more output signals conveying information related to one or more gas parameters of the pressurized flow of breathable gas being delivered to the airway of the subject; and
determining one or more CO₂ parameters based on the one or more generated output signals during the first therapy mode and the second therapy mode.

10. The method of claim 9, further comprising:
determining one or more cardiac output parameters based on the one or more determined CO₂ parameters for partial CO₂ rebreathing.

11. A system configured to provide ventilation to an airway of a subject through a ventilation system, the system comprising;
means for generating a pressurized flow of breathable gas for delivery to the airway of the subject;
delivery means guiding the pressurized flow of breathable gas to the airway of the subject;
means for determining whether the ventilation system is operating in a first therapy mode or a second therapy mode;
first exhaust means for selectively exhausting gas from the delivery circuit primarily via a first exhaust point, responsive to a determination that the ventilation system is operating in the second therapy mode; and
means for selectively exhausting gas from the delivery circuit primarily via a second exhaust point, responsive to a determination that the ventilation system is operating in the first therapy mode, wherein the delivery means has a rebreathing storage capacity,
wherein operation of the first exhaust means increases a CO₂ rebreathing volume of the subject and the delivery circuit by the rebreathing storage capacity.

12. The system of claim 11, further comprising:
means for determining whether a current respiratory phase of the subject is an inhalation phase or an exhalation phase,
wherein operation of the first exhaust means and operation of the second exhaust means is furthermore responsive to a determination that the current respiratory phase of the subject is not an inhalation phase.

13. The system of claim 11, wherein the first exhaust means is operated during inhalation phases and exhalation phases during the second therapy mode.

14. The system of claim 11, further comprising:
means for generating one or more output signals conveying information related to one or more gas parameters of the pressurized flow of breathable gas being delivered to the airway of the subject; and
means for determining one or more CO₂ parameters based on the one or more generated output signals during the first therapy mode and the second therapy mode.

15. The system of claim 14, wherein the means for determining one or more CO₂ parameters furthermore determines one or more cardiac output parameters based on the one or more determined CO₂ parameters for partial CO₂ rebreathing.
